(19) United States
(12) Patent Application Publication
(10) Pub. No.: US 2021/0214808 A1
(43) Pub. Date: Jul. 15, 2021

(54) A KIT FOR THE SPECIFIC DETECTION OF TRICHOMONAS TENAX, A SET OF PRIMERS FOR THE SPECIFIC DETECTION OF TRICHOMONAS TENAX AND A METHOD FOR THE SPECIFIC DETECTION OF TRICHOMONAS TENAX

(71) Applicant: UNIVERSIDAD DE ANTOFAGASTA, Antofagasta (CL)

(72) Inventors: Jorge Enrique Araya Rojas, Antofagasta (CL); Alejandro Catalan Rodriguez, Antofagasta (CL); Casandra Bracamonte Wolf, Antofagasta (CL); Benito Gomez Silva, Antofagasta (CL)

(21) Appl. No.: 17/053,771

(22) PCT Filed: May 9, 2019
(86) PCT No.: PCT/IB19/53835

§ 371 Date: Nov. 7, 2020

Related U.S. Application Data
(60) Provisional application No. 62/668,987, filed on May 9, 2018.

(51) Int. Cl. C12Q 1/6893; C12Q 1/686; C12Q 1/6806

(52) U.S. Cl. CPC C12Q 1/6893; C12Q 1/6806; C12Q 1/686

(57) ABSTRACT

The present invention relates to a detection kit, a set of primers contained in said kit and a method of using the kit for the specific detection of T. tenax that can be used to the specific detection, prevention and/or infectious disease control associated with Trichomonas tenax in humans. The kit, the primers and the method of use described herein allow the specific detection of T. tenax by identifying a single nucleotide polymorphism of the gene that encoding β-tubulin of T. tenax in a subgingival or pulmonary exudate sample, allowing to differentiate if the infection is produced by T. tenax or T. vaginalis.

### SEQUENCE LISTING

The sequence listing submitted via EFS, in compliance with 37 CFR § 1.52(e)(5), is incorporated herein by reference. The sequence listing text file submitted via EFS contains the file “SEQUENCE LISTING WO2019215667.txt”, created on Nov. 7, 2020, which is 723 bytes in size.

### FIELD OF THE INVENTION

The present invention is related to the field of molecular biology, immunology and infectology. More specifically, the invention relates to the diagnostic, prevention and/or infectious disease control associated with Trichomonas tenax.

In particular, the invention relates to a detection kit, a set of primers contained in said kit and a method of using the kit for the specific detection of T. tenax, by means of the identification of a single nucleotide polymorphism of the gene that codifies for β-tubulin of T. tenax in a subgingival sample or pulmonary exudate, allowing to differentiate if the infection is produced by T. tenax or T. vaginalis.

### STATE OF ART

Trichomonas tenax is a flagellated anaerobic protozoan considered for a long time a commensal organism that inhabits the human oral cavity. This microorganism belongs to the genus Trichomonas, which also includes Trichomonas vaginalis, another human host species.

T. tenax has been found in patients with poor oral hygiene and periodontal disease. Periodontal disease is considered as a wide range of inflammatory conditions that affect the supporting structures of the teeth. This disease begins with gingivitis, a reversible localized periodontal inflammatory disease, which can progress to chronic periodontitis, characterized by an irreversible loss of tooths, bones and ligaments.

The prevalence of this microorganism in periodontal disease is heterogeneous, oscillating between 0 and 94%, This can be attributed to an unspecific detection due to a low sensitivity and specificity diagnostics methods.

The conventional approach for detecting infection with T. tenax is to perform direct microscopic examination, with or without staining, and the culture of dental plaque samples. Both methods have disadvantages associated to the excessive time used and low sensitivity and specificity, being insufficient to discriminate between different Trichomonas species, requiring specific technical skills and experience with highly qualified personnel for their correct detection.

Advances in molecular biology have brought new detection methods more faster, sensitive and reliable for the specific detection of microorganisms, for example the polymerase chain reaction (PCR).

PCR methods for the detection of T. tenax are based on the amplification of the 18S rRNA gene or 5,8S rRNA. However, these sequences are not widely available in clinical laboratories, which hinders their access to this type of diagnosis and sometimes the sequencing of the amplified sequences is necessary, extending the diagnostic time even further.

A methods, reagents and kits for determining the presence of Trichomonas vaginalis in a test sample was disclosure in the document WO2012075321. The embodiments of the invention comprise at least one primer pair comprising a forward and reverse primer capable of hybridizing to a target region of Trichomonas vaginalis using the gene TV5.8S as a target.

Another example associated with the detection of Trichomonas by molecular biology is disclosed in WO2015114368, which disclose a method for determining the presence or absence of T. vaginalis through the detection of ribosomal protein TV40S by a PCR methodology.

Although, the molecular biology-based methodologies such as PCR or qPCR described in documents WO2012075321 and WO2015114368 allow to accelerate the detection times of these microorganisms with greater sensitivity and specificity, they can not discriminate between T. vaginalis and T. tenax in a single trial, due to its close morphological and DNA level identity. In contrast to these, the present invention described here proposes a methodology based on molecular biology, specifically a polymerase chain reaction (PCR) assay for the amplification of the T. tenax β-tubulin gene, followed by the screening of a single nucleotide polymorphism (SNP) of the amplified T. tenax β-tubulin gene by restriction fragment length polymorphism (RFLP) assay to the discrimination between T. tenax and T. vaginalis species. This kind of invention is interest for the molecular diagnostic industry and infectious disease control in the light of the prior art.

### SUMMARY DESCRIPTION OF THE INVENTION

The present invention relates to a detection kit, a set of primers contained in said kit and a method of using the kit for the specific detection of T. tenax that can be used to the specific detection, prevention and/or infectious disease control associated with Trichomonas tenax in humans. The kit, the primers and the method of use described herein allow the specific detection of T. tenax by identifying a single nucleotide polymorphism of the gene that encoding β-tubulin of T. tenax in a subgingival or pulmonary exudate sample, allowing to differentiate if the infection is produced by T. tenax or T. vaginalis.

### BRIEF DESCRIPTION OF THE FIGURES

A PCR reaction of the β-tubulin gene for detection of T. tenax is shown in FIG. 1. Specifically, FIG. 1.A shows the specificity of the PCR reaction of the β-tubulin gene; the sensitivity of the PCR reaction of the β-tubulin gene is shown in FIG. 1.B; and in FIG. 1.C the detection limit T. tenax is illustrated. M: 100 bp molecular ladder marker; Tt: Trichomonas tenax; Tv: Trichomonas vaginalis; Pg: Porphyromonas gingivalis; Aa: Aggregatibacter actinomycetemcomitans; Hs: Homo sapiens; Neg: Water.

FIG. 2 shows a PCR-RFLP reaction for differentiation between T. tenax and T. vaginalis. Specifically, FIG. 2.A shows the partial multiple alignment of nucleotide sequences between the β-tubulin gene sequence of T. tenax and the β-tubulin gene sequences of T. vaginalis, the restriction sequence for the NdeI enzyme is shown in black, different nucleotides are shown between the sequences, the accesses of the sequences used are shown on the left side; while FIG. 2.B shows the enzymatic digestion of PCR product of Trichomonas tenax (T.t) and Trichomonas vaginalis (T.v) with NdeI.

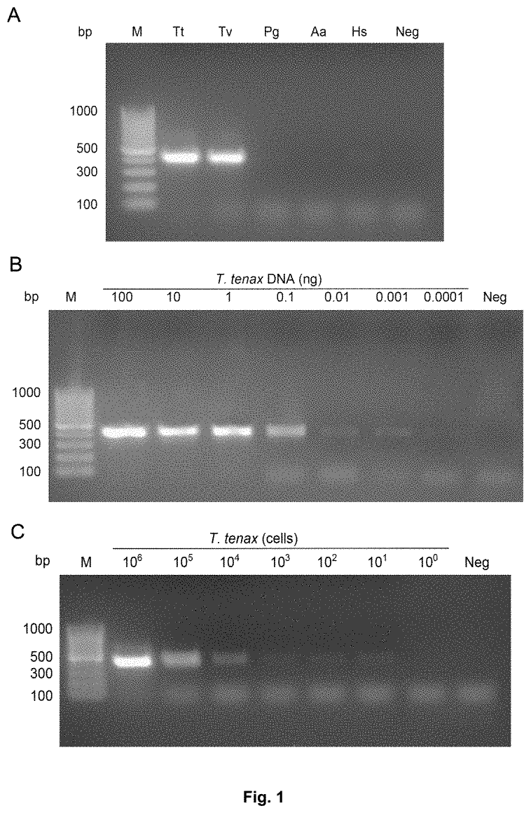

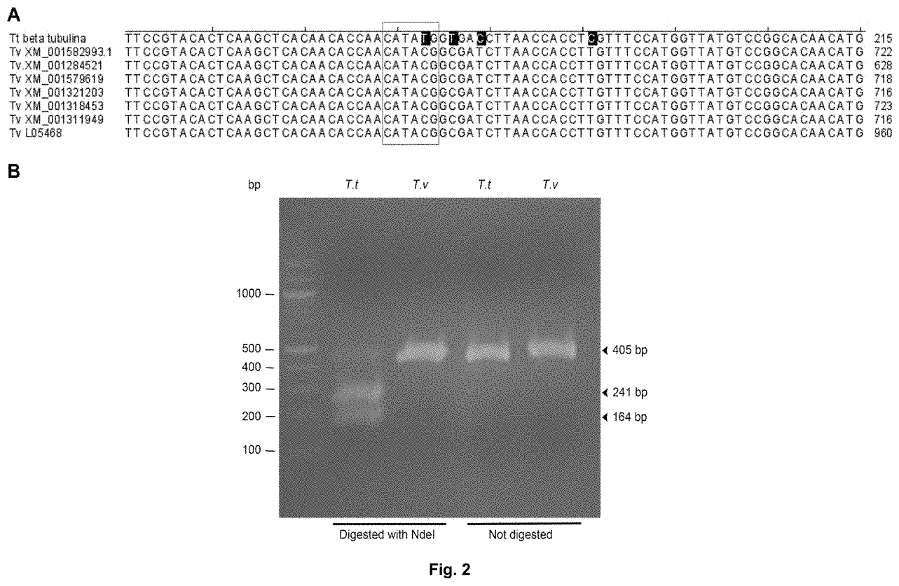

### DETAILED DESCRIPTION OF THE INVENTION

Trichomonas tenax and Trichomonas vaginalis are flagellated protozoans of the genus Trichomonas that cause parasitic infections in humans.

T. vaginalis inhabits the urogenital tract and is the most prevalent non-viral sexually transmitted infection agent worldwide, a disease known as trichomoniasis, which globally reports 143 million new cases annually. In women, this non-viral sexually transmitted disease causes severe clinical symptoms, including complications in pregnancy, cervical neoplasia, atypical pelvic inflammatory disease and risks of infertility. In men, the clinical symptoms presented by this infection range from non-gonococcal urethritis, prostatitis, epididymitis and even risks of infertility.

T. tenax, however, lives in the oral cavity of people with poor oral hygiene, being detected in recent years in patients with periodontal disease.

T. tenax has long been considered a commensal protozoan that inhabits the human oral cavity, however, it has been reported that this microorganism meets the requirements to be considered a protozoan parasite, given its ability to damage different cells epithelial mammal, behaving very similar to T. vaginalis.

Although the T. vaginalis species has been found mainly in the vaginal cavity, it has also been detected in the oral cavity of pregnant women and sex workers, while, in turn, T. tenax has been detected in vaginal samples.

Currently, both T. tenax and T. vaginalis have been associated with bronchopulmonary infections in patients with Pneumocystis carinii or with underlying cancers or other pulmonary diseases. Moreover, it has been reported that both species of trichomonas have entered the respiratory tract by aspiration from the oropharynx, so the origin of the bronchopulmonary infection may be uncertain.

The latter reinforces the importance of a differentiation of species when amplification methods are made from DNA extracted from the oral cavity. In this sense, the present invention proposes a detection kit, the primers contained in said kit and a method of using the kit for the specific detection of T. tenax, by means of the identification of a single nucleotide polymorphism of the gene coding for β-tubulin of T. tenax in a subgingival sample or pulmonary exudate, allowing to differentiate if the infection is caused by T. tenax or T. vaginalis.

The β-tubulin protein is essential in the morphology of different protozoa, forming, together with α-tubulin, the structure of microtubules, very abundant structures in the genus Trichomonas. The identity between the β-tubulin sequences of T. tenax and T. vaginalis corresponds to 94-96%, which demonstrates their high degree of genetic identity.

However, there is a single nucleotide mutation (SNP) in nucleotide 168 of the β-tubulin gene of T. tenax, corresponding to a substitution of T for C, which is not present in T. vaginalis. This mutation generates a restriction site for the NdeI enzyme, which can convert the 405 bp sequence corresponding to the β-tubulin gene of T. tenax into two digestion fragments of 241 bp and 164 bp each, while in T. vaginalis this enzymatic cut does not occur, being able to discriminate in an accurate way between both species.

For that, the present invention proposes, a kit proposed in the present invention comprises, at least:

A.—a forward primer corresponding to SEQ ID No. 1 of 90% to 100% identity;
B.—a reverse primer corresponding to SEQ ID No. 2 of 90% to 100% identity; and
C.—a restriction enzyme.

Where the primers for the amplification of the β-tubulin gene of Trichomonas possess the following sequences:

| | |
|---|---|
| SEQ ID n° 1 | 5′-ATACTCTATCGTCCCATCTC-3′ |
| SEQ ID N° 2 | 5′-GCCATCATGTTCTTGTTATCG-3′ |

The product of this amplification corresponds to a sequence of 405 bp for both Trichomonas species.

While the restriction enzyme corresponds to NdeI, which performs a specific enzymatic cleavage in the sequence 5′-CA∇TATG-3.

The different components within the kit are in an optimum concentration range. Finding the concentration of the forward or forward splitter in a range of 100 to 500 nM, preferably in a concentration of 250 to 450 nM, more preferably 400 nM.

The concentration of the antisense or reverse party is in a range of 100 to 500 nM, preferably in a concentration of 250 to 450 nM, more preferably 400 nM.

For the case of the enzyme it corresponds to the restriction enzyme NdeI, which is diluted in a buffer in a concentration of 1 to 40 U/μL, preferably in a concentration of 10 to 25 U/μL, more preferably 20 U/μL, wherein the buffer consists of a buffer containing 50 mM Tris-HCl (pH 7.5), 10 mM MgCl₂, 100 mM NaCl and 0.1 mg/mL bovine serum albumin.

Alternatively, the kit may comprise a reagent or base solution comprising of PCR reaction buffer (Tris-HCl pH 8.8; (NH₄)₂SO₄; Tween 20); dNTPs; MgCl₂ and Taq DNA polymerase.

The present invention describes a primer for the specific detection of Trichomonas tenax, wherein it comprises a sequence corresponding to SEQ ID No. 1 of 90% to 100% identity; and a primer for the specific detection of Trichomonas tenax, wherein it comprises a sequence corresponding to SEQ ID No. 2 of 90% to 100% identity.

Additionally, the method for the specific detection of Trichomonas tenax proposed in the following invention comprises the following steps:
i. extract DNA from a sample from the affected area of the subject;
ii. amplify the DNA obtained in the previous step by PCR using the following primers:
   A.—a forward primer corresponding to SEQ ID No.1 of 90% to 100% identity;
   B.—a reverse primer corresponding to SEQ ID No.2 of 90% to 100% identity;
iii. digest the amplified DNA obtained in the previous step using a restriction enzyme;
iv. submit the fragments of the digestion to an electrophoresis gel;
v. discriminate Trichomonas tenax by comparing the result of electrophoresis obtained in the previous stage with a reference standard, corresponding to T. tenax ADN previously digested with NdeI enzyme.

For step (i) of extracting DNA from a sample from the affected area of the subject, the procedure to be performed is as follows:
a) taking the sample of the subject from the oral cavity and/or from the saliva and sputum and/or form the bronchoalveolar fluid and/or from the pleural exudate;
b) extract the genomic DNA from the sample obtained in the previous step.

For sample taking from the oral cavity, a mouth swab extraction device is rubbed on the dental surface for 3 to 7 seconds.

For sampling from the saliva and sputum a volume between 1 to 5 mL will be require.

For sample taking from bronchoalveolar fluid, will be taken by bronchoalveolar lavage.

For sample taking pleural exudate, pleural fluid will be taken by thoracocentesis.

For extracting DNA from the sample obtained from the subject, the sample is first subjected to a cell lysis, which is selected from a chemical method and a physical method. Next, the sample subjected to a cell lysis is washed with detergents for the removal of the membrane lipids. Then the previous sample is subjected to a treatment with proteases for the denaturation and removal of proteins. The RNA is removed by treating the supernatant sample with RNase enzymes. The sample is centrifuged at each stage, and the supernatant is maintained. Finally eluting the DNA in slightly alkaline buffer and distilled water.

For step (ii) of DNA amplification by PCR, the procedure to be performed is as follows:
a) adding in a microtube the following: the DNA extracted in step (i) above; the forward primer SEQ ID No.1 and reverse primer SEQ ID No. 2; PCR buffer (Tris-HCl pH 8.8; (NH₄)₂SO₄; Tween 20); dNTPs; MgCl₂ and Taq DNA polymerase.
b) perform the PCR reaction in a thermocycler.

In that the PCR reaction is performed in a thermocycler, using the following thermal profile:

| Steps | Temperature (° C.) | Time | |
|---|---|---|---|
| Initial denaturation | 95 | 3 | min |
| Denaturation | 95 | 30 | sec |
| Annealing | 40-50 | 20-60 | sec |
| Elongation | 72 | 1 | min |
| Final step | 72 | 7 | min |

In preferential way, the PCR reaction is performed in a thermocycler for a total of 35 cycles, using the following thermal profile:

| Cycles | Steps | Temperature (° C.) | Time | |
|---|---|---|---|---|
| 1 | Initial denaturation | 95 | 3 | min |
| 35 | Denaturation | 95 | 30 | sec |
| | Annealing | 46-47 | 20 | sec |
| | Elongation | 72 | 1 | min |
| 1 | Final step | 72 | 7 | min |

In step (iii) of digestion of the amplified DNA, the following procedure is performed:

a) Take a volume of the amplificated product by PCR obtained in the previous step, add the restriction enzyme, wherein the restriction enzyme is NdeI, until reaching a volume ratio amplified product: enzyme of 1:3, where the enzyme is diluted in a buffer, in where the buffer comprises 50 mM Tris-HCl (pH 7.5), 10 mM MgCl₂, 100 mM NaCl and 0.1 mg/mL bovine serum albumin.
b) Incubate the mixture obtained in the previous step (a), at a temperature between 20 to 50° C. and for between 1 to 30 hours to, preferably at 37° C. for 16 hours.

For step (iv) the enzymatic digestion products with NdeI obtained in step (iii) are separated by a gel electrophoresis between 0,6 to 2% agarose gel, preferably in 1.5% agarose gel and visualized by ethyl bromide (BrEt) staining.

In the method describe above, the mixture obtain in the step (iii) is incubated at a temperature of 37° C. for 16 hours.

Further, as a result of the digestion of the β-tubulin of Trichomonas tenax two DNA fragments are produced in the step (iii) of the method, wherein the DNA fragment 1 comprises a size of 200 bp to 300 bp and the DNA fragment 2 comprises a size from 100 bp to 200 bp.

Finally, for stage (v) of discrimination of Trichomonas tenax by comparing the result of electrophoresis obtained in the previous stage with a reference standard (T. tenax ADN previously digested with NdeI enzyme).

For this, the gel image is photo-documented using a gel documentation system. This image is then compared with a standard reference available in the kit set

Likewise, the molecular weight can be checked according to the molecular weight markers that are placed on the side of this gel resulting from electrophoresis.

### EXAMPLES OF APPLICATION

### Example 1

Extraction of DNA from a Sample from the Affected Area of the Subject:

Fifty individuals were selected, of both genders with ages between 20 and 80 years. Individuals were classified into 2 groups: 1) Adult individuals with plaque-induced gingivitis (20 patients); 2) Adult individuals with severe generalized chronic periodontitis (30 patients). Bacterial plaque samples from patients with severe chronic generalized periodontitis (PCGS) were obtained through a cone of gutta-percha, previously disinfected with 0.12% chlorhexidine, which was introduced subgingivally, and rubbed for 5 seconds on the tooth surface. A dental calculus sample was obtained using a sterile curette. Both samples were obtained from periodontal sacs with a depth of probing 5 mm and deposited in a microtube with 1.5 mL of sterile Ringer's solution.

Bacterial plaque and dental calculus samples from patients with gingivitis were obtained with a sterile curette. Both samples were deposited in a microtube with 1.5 mL of sterile Ringer's solution.

The extraction of genomic DNA from clinical samples of the oral cavity were performed using the inorganic phenol-chloroform method. For the extraction of DNA from subgingival plaque samples, the samples were centrifuged at 800×g for 10 minutes at 4° C. Then, the cell pellet was washed 3 times in sterile PBS, pH 7.2. The cells were lysed with 500 μL of TELT lysis buffer (50 mM Tris-HCl, pH 8.0, 4%, Triton X-100, 62.5 mM EDTA pH 8.0, and 2.5 M LiCl) with 1 mg/mL of RNAse A and incubated at 37° C. for 30 minutes, followed by 100 μg/mL Proteinase K and incubated at 50° C. for 2 hours. Then, the same volume of phenol-chloroform-isoamyl alcohol (25:24:1) was added. The suspension was vigorously stirred and centrifuged at 13,000×g for 10 minutes at 4° C. The aqueous phase was collected and transferred to a new eppendorf tube, then an equal volume of chloroform was added, and it was centrifuged at 13,000×g for 10 minutes at 4° C. The aqueous phase was recovered and transferred to a new eppendorf tube. The DNA in solution was precipitated with 0.3 M sodium acetate pH 5.5 and 2.5 times the volume of cold absolute ethanol. The sample was centrifuged at 13,000×g for 20 min at 4° C. The supernatant was discarded, and the precipitate was washed with 500 μL of 70% ethanol and then centrifuged at 13,000×g for 5 minutes at 4° C. The precipitate was dried at 37° C. for 10 minutes. Finally, the precipitate was resuspended in 50 μL of free-nuclease water.

### DNA Amplification by PCR:

The PCR reaction was performed for a 50 μL reaction volume, containing: 1 μL DNA, 75 mM Tris-HCl pH 8.8, 20 mM (NH₄) 2 SO₄, 0.01% (v/v) Tween 20, 0.2 mM of each dNTPs, 2.5 mM of MgCl₂, 400 nM of each forward primer SEQ ID No. 1 and reverse SEQ ID No. 2, and 2.5 U of Taq DNA polymerase. The reaction was performed in a thermocycler for a total of 35 cycles, using the following reaction conditions: denaturation at 95° C. for 3 minutes, followed by 35 cycles of denaturation at 95° C. for 30 seconds, annealing at 46.5° C. for 20 seconds, elongation at 72° C. for 1 minute, and a final stage of 72° C. for 7 minutes. The amplification products were separated by electrophoresis in 1% agarose gel and stained with ethidium bromide (BrEt). Images of the gels were photo-documented using a MF-ChemiBIS 2.0 gel documentation system (DNR Bio-Imaging Systems, Mahale HaHamisha, Jerusalem, Israel), using the Gel Capture Pro software.

### Digestion of the Amplified DNA:

The amplification products (10 μL) were digested with the NdeI enzyme dissolved in a buffer containing bovine serum albumin for a reaction volume of 30 μL and incubated at 37° C. for 16 hours.

### Elution of the Restriction Fragments in Gel Electrophoresis:

The restriction fragments obtained from the fragmentation of the DNA by the restriction enzyme NdeI were analyzed using a gel electrophoresis in 1.5% agarose gel and visualized by staining with bromide ethyl (BrEt).

T. tenax Discrimination:

The gel image was photo-photographed using a MF-ChemiBlS 2.0 gel documentation system (DNR Bio-Imaging Systems, Mahale HaHamisha, Jerusalem, Israel), using the Gel Capture Pro software. This image was then compared with a reference pattern obtained by the same methodology.

1. A set of primers for the detection of Trichomonas tenax, said set comprises:
a forward primer corresponding to SEQ ID No. 1 of 90% to 100% identity; and
a reverse primer corresponding to SEQ ID No. 2 of 90% to 100% identity.

2. A kit for the detection of Trichomonas tenax, said kit comprises:
a forward primer corresponding to SEQ ID No. 1 of 90% to 100% identity;
a reverse primer corresponding to SEQ ID No. 2 of 90% to 100% identity; and
a restriction enzyme.

3. The kit according to claim 2, wherein a concentration of the forward primer ranges from 100 nM to 500 nM.

4. The kit according to claim 2, wherein a concentration of the forward primer ranges from 250 nM to 450 nM.

5. The kit according to claim 2, wherein a concentration of the forward primer is 400 nM.

6. The kit according to claim 2, wherein a concentration of the reverse primer ranges from 100 nM to 500 nM.

7. The kit according to claim 2, wherein a concentration of the reverse primer ranges from 250 nM to 450 nM.

8. The kit according to claim 2, wherein a concentration of the reverse primer is 400 nM.

9. The kit according to claim 2, wherein a concentration of the restriction enzyme ranges from 1 to 40 U/μL.

10. The kit according to claim 2, wherein a concentration of the restriction enzyme ranges from 10 to 25 U/μL.

11. The kit according to claim 10, wherein a concentration of the restriction enzyme is 20 U/μL.

12. The kit according to claim 2, wherein the restriction enzyme corresponds to NdeI.

13. The kit according to claim 2, wherein the kit further comprises PCR buffer; dNTPs set; MgCl₂ and Taq DNA polymerase.

14. A method for the detection of Trichomonas tenax comprising the steps of:
amplifying DNA from a sample by PCR using the following set of primers:
a forward primer corresponding to SEQ ID No.1 of 90% to 100% identity; and
a reverse primer corresponding to SEQ ID No.2 of 90% to 100% identity;
digesting the amplified DNA using a restriction enzyme;
submitting the fragments of the digestion to an electrophoresis gel; and
discriminating Trichomonas tenax by comparing the result of the electrophoresis with a reference standard.

15. The method according to claim 14, wherein said sample is from an affected area of a subject and said DNA is extracted by:
taking the sample of the subject from at least one of: the oral cavity or the saliva and sputum or the bronchoalveolar fluid or the pleural exudate; and
extracting the genomic DNA from the sample.

16. The method according to claim 15, wherein a mouth swab extraction device is rubbed on a dental surface for 3 to 7 seconds when the sample is taken from the oral cavity.

17. The method according to claim 15, wherein the sample taken from the saliva and sputum has a volume between 1 to 5 mL.

18. The method according to claim 15, wherein the sample from the bronchoalveolar fluid is taken by bronchoalveolar lavage.

19. The method according to claim 15, wherein the sample from the pleural exudate is taken by thoracocentesis.

20. The method according to claim 15, wherein said DNA is extracted from the sample by:
subjecting the sample to a cell lysis, which is selected from a chemical method and a physical method;
washing the sample subjected to the cell lysis is for the removal of membrane lipids, wherein the sample is centrifuged, and supernatant is maintained;
subjecting the previous sample to a treatment with proteases for the denaturation and removal of proteins;
removing RNA by treating the supernatant sample with RNase enzymes, wherein the sample is centrifuged, and the supernatant is maintained; and
eluting the DNA in slightly alkaline buffer and distilled water.

21. The method according to claim 14, wherein said DNA is amplified by:
adding in a microtube the extracted DNA; the forward primer SEQ ID No. 1; the reverse primer SEQ ID No.2; PCR buffer; dNTPs; MgCl₂ and Taq DNA polymerase; and
performing the PCR reaction in a thermocycler.

22. The method according to claim 21, wherein the PCR reaction is performed in a thermocycler, using the following thermal profile:

| Steps | Temperature (° C.) | Time | |
|---|---|---|---|
| Initial denaturation | 95 | 3 | min |
| Denaturation | 95 | 30 | seg |
| Annealing | 46-47 | 20-60 | seg |
| Elongation | 72 | 1 | min |
| Final step | 72 | 7 | min |

23. The method according to claim 22, wherein the thermal profile comprises 35 cycles, according to:

| Cycles | Steps | Temperature (° C.) | Time | |
|---|---|---|---|---|
| 1 | Initial denaturation | 95 | 3 | min |
| 35 | Denaturation | 95 | 30 | seg |
| | Annealing | 46-47 | 20-60 | seg |
| | Elongation | 72 | 1 | min |
| 1 | Final step | 72 | 7 | min |

24. The method according to claim 14, wherein the forward primer and the reverse primer amplify a coding sequence for trichomonas β-tubulin.

25. The method according to claim 14, wherein the amplified DNA is digested by:
adding a restriction enzyme to a volume of the amplificated product by PCR until reaching an amplified product:enzyme volume ratio of 1:3, where the restriction enzyme is diluted in a buffer comprising bovine serum albumin; and
incubating a mixture obtained in the previous step, at a temperature between 20 to 50° C. and for between 1 to 30 hours.

26. The method according to claim 25, wherein the buffer comprises at least 0.1 mg/mL of said bovine serum albumin.

27. The method according to claim 14, wherein the restriction enzyme corresponds to NdeI.

28. The method according to claim 25, wherein the mixture obtain is incubated at a temperature of 37° C. for 16 hours.

29. The method according to claim 14, wherein two DNA fragments are produced as a result of digesting the β-tubulin gene of Trichomonas tenax, a first DNA fragment comprising a size of 200 bp to 300 bp and a second DNA fragment 2-comprising a size from 100 bp to 200 bp.

30. The method according to claim 14, wherein the fragments of the digestion submitted to said electrophoresis gel are separated by a gel electrophoresis which comprises between 0.6 to 2% agarose gel and visualized by ethyl bromide (BrEt) staining.

31. The method according to claim 30, wherein the gel electrophoresis comprises 1.5% agarose gel.

32. The method according to claim 14, wherein said Trichomonas tenax is discriminated by comparing the result of the electrophoresis gel against a reference digestion pattern DNA of Trichomonas tenax.

33. The method according to claim 14, wherein said Trichomonas tenax is discriminated by checking a molecular weight according to molecular weight markers that are placed on ha side of the gel resulting from electrophoresis.


SEQUENCE LISTING (2 sequences)

SEQ ID NO: 1 - DNA, 20 bp, Artificial Sequence
  PCR_primer
atactctatcgtcccatctc

SEQ ID NO: 2 - DNA, 21 bp, Artificial Sequence
  PCR_primers
gccatcatgttcttgttatcg